(19) United States
(12) Patent Application Publication
(10) Pub. No.: US 2011/0224438 A1
(43) Pub. Date: Sep. 15, 2011

(54) NOVEL PROCESS FOR THE MANUFACTURE OF 5-HALOGENATED-7-AZAINDOLES

(76) Inventors: Hongjun Gao, Hangzhou City (CN); Stefan Hildbrand, Gelterkinden (CH); Christoph Hoefler, Rheinfelden (DE); Wenfa Ye, Taizhou City (CN); Guoliang Zhu, Taizhou City (CN)

(21) Appl. No.: 13/039,383

(22) Filed: Mar. 3, 2011

(30) Foreign Application Priority Data
Mar. 9, 2010 (CN) PCT/CN2010/070925

(51) Int. Cl. C07D 471/04

(52) U.S. Cl. 546/113

(57) ABSTRACT

The present invention provides a novel method for manufacturing the compound of formula (I)

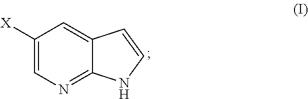

wherein X is —Cl or —Br.

### PRIORITY TO RELATED APPLICATION(S)

This application claims the benefit of International Patent Application No. PCT/CN2010/070925, filed Mar. 9, 2010, which is hereby incorporated by reference in its entirety.

### FIELD OF THE INVENTION

The present invention relates to a new process for the manufacture of 5-bromo-7-azaindole (CAS 183208-35-7) or 5-chloro-7-azaindole (CAS 866546-07-8) of formula (I).

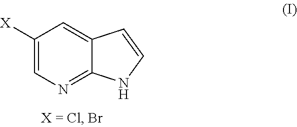

The compounds of general formula (I) are valuable starting materials in the synthesis of more complex heterocyclic molecules, which may be used in many different commercial products, and inter alia as medicaments.

### SUMMARY OF THE INVENTION

The present invention provides a process for the manufacture of the compound of formula I

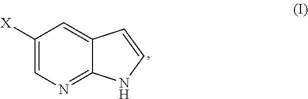

wherein,

(a) the compound of formula (II)

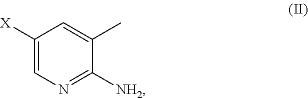

is reacted in the presence of the compound of formula (III)

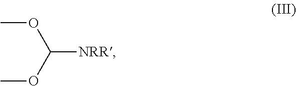

to give the compound of formula (IV)

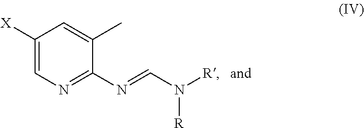

(b) said compound of formula (IV) is further reacted in the presence of a strong base to give the compound of formula (I), and wherein

R and R′ are each independently C1-4 alkyl; and

### X is —Cl or —Br.

### DETAILED DESCRIPTION OF THE INVENTION

Current methods for the synthesis of the compounds of formula (I) are disclosed in US 2006/0183758 and WO03/082869. The disclosed processes are multistep synthesis routes which use environmentally hazardous reagents and/or require the use of heavy metals which are difficult to remove in the subsequent steps.

It is therefore an object of the present invention to provide a process for manufacturing the compounds of formula (I) which uses few reaction steps, in particular 2 steps, and less hazardous reagents. The present process is thus particularly useful in large scale manufacturing of the compounds of formula (I).

The present invention provides a process for the manufacture of the compound of formula I

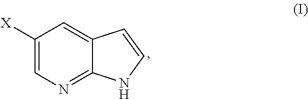

wherein,

(a) the compound of formula (II)

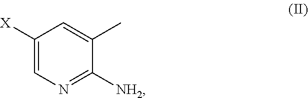

is reacted in the presence of the compound of formula (III)

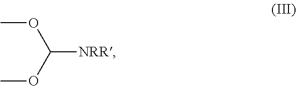

to give the compound of formula (IV)

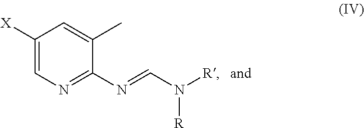

(b) said compound of formula (IV) is further reacted in the presence of a strong base to give the compound of formula (I), and wherein

R and R′ are each independently C1-4 alkyl; and

### X is —Cl or —Br.

The term “strong base” as used herein means strong organic bases like for example alkali metal amides, in particular lithium diisopropylamide (LDA), n-butyl lithium (n-BuLi).

The term “C1-4 alkyl” means a saturated, linear or branched hydrocarbon containing from 1 to 4 carbon-atoms. An especially preferred group is the methyl group.

The reaction step (a) as disclosed herein is preferably carried out using a solvent selected from heptane, toluene, xylene, dimethylsulfoxide (DMSO), dimethylformamide (DMF), chlorobenzene, o-dichlorobenzene, p-dichlorobenzene, m-dichlorobenzene, 2-methyltetrahydrofuran, N-methyl-2-pyrrolidone. The reaction may be carried out at temperatures within the range of 60 to 120° C.

The reaction step (b) as disclosed herein is preferably carried out using a solvent selected from diethyl ether, methyl t-butyl ether, n-hexane and tetrahydrofuran (THF). The reaction may be carried out at temperatures ranging from −85 to 30° C.

The term “strong base” as used in reaction step (b) herein means lithium diethylamide, potassium diisopropylamide (KDA), lithium hexamethyldisilazide (LHMDS), Sodium hexamethyldisilazide (NaHMDS), n-butyl lithium (n-BuLi), s-Butyl lithium (s-BuLi), t-butyl lithium (t-BuLi), lithium isopropyl cyclohexylamide (LICA), lithium 2,2,6,6-tetramethylpiperidide (TMPLi), ((trimethylsilyl)methyl)lithium (TMSCH₂Li), lithium di-(trimethylsilyl)methyl amide (TMS₂CHLi), and the like.

In an embodiment of the present invention,

step (a) is carried out using a solvent selected from heptane, toluene, xylene, dimethylsulfoxide (DMSO), dimethylformamide (DMF), chlorobenzene, o-dichlorobenzene, p-dichlorobenzene, m-dichlorobenzene, 2-methyltetrahydrofuran and N-methyl-2-pyrrolidone, and at a temperature within the range of 60 to 120° C.; and
step (b) is carried out using a solvent selected from diethyl ether, methyl t-butyl ether, n-hexane and tetrahydrofuran (THF), a reaction temperature in the range from −85 to 30° C., and a strong base selected from lithium diethylamide, potassium diisopropylamide (KDA), lithium hexamethyldisilazide (LHMDS), sodium hexamethyldisilazide (NaHMDS), n-butyl lithium (n-BuLi), s-Butyl lithium (s-BuLi), t-butyl lithium (t-BuLi), lithium isopropyl cyclohexylamide (LICA), lithium 2,2,6,6-tetramethylpiperidide (TMPLi), ((trimethylsilyl)methyl)lithium (TMSCH₂Li) and lithium di-(trimethylsilyl)methyl amide (TMS₂CHLi).

In a particularly preferred embodiment of the present invention, X is —Br (compound 1).

In another particularly preferred embodiment of the present invention, X is —Cl (compound 1a).

In still another preferred embodiment of the present invention, X is —Br; R and R′ are both methyl, and the strong base in reaction step (b) is LDA.

In still another preferred embodiment of the present invention, X is —Cl; R and R′ are both methyl, and the strong base in reaction step (b) is LDA.

A particularly preferred embodiment according to the present invention is the synthesis of the compound of formula (I) wherein X is —Br, starting from the compound of formula (2) (see below) and using the specific reaction conditions described in Examples 1 and 2a below.

Another particularly preferred embodiment according to the present invention is the synthesis of the compound of formula (I), wherein X is —Br, starting from the compound of formula (2) (see below) and using the specific reaction conditions described in Examples 1 and 2b below.

The invention is now illustrated by the accompanying working examples, which are not meant to limit the scope of the present invention.

### EXAMPLES

### Example 1

### Synthesis of N-(5-bromo-3-methyl-pyridin-2-yl)-N,N-dimethyl-formamidine (4)

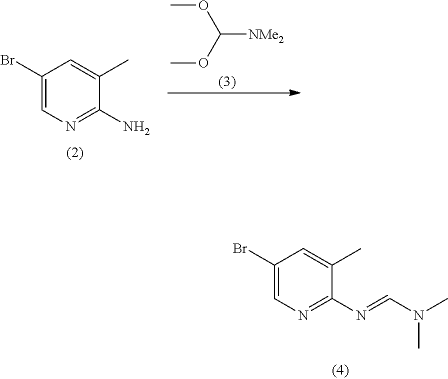

A mixture of 37.4 g (0.20 mol) of 2-amino-5-bromo-3-methylpyridine and 33 mL (0.25 mol) of N,N-dimethylformamide dimethyl acetal (3) was heated to reflux temperature for about 15 h (until the disappearance of the starting material was indicated by HPLC analysis). The reaction mixture was cooled and then concentrated under vacuum (35-40 mmHg) at 55° C. to constant weight. Heptane (45 mL) was added in one portion and the mixture stirred for 30 min. The mixture was cooled to −20° C. and then kept at this temperature for 5 h for crystallization. The crystals were filtered, washed with 30 g of cold heptane and dried under vacuum at room temperature to afford 44.55 g (92%) of compound 4 with a purity of >99 area % (HPLC).

### Example 2a

### Synthesis of 5-bromo-7-azaindole (1)

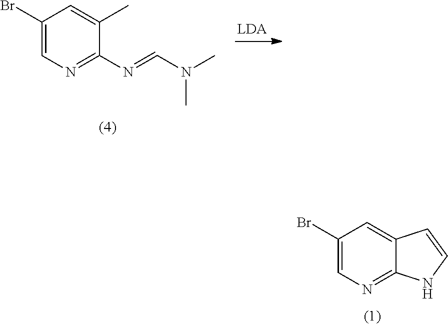

A four-necked flask fitted with a thermometer, adding funnel, and mechanical stirring was flushed with nitrogen for 3 times before use. To this flask, 75 mL of LDA (2.0 M in THF, 0.15 mol) was added and then cooled to −30±2° C. In a separate vessel, 24.2 g (0.10 mol) of compound (4) was dissolved in 121 mL of dry THF and then the solution was injected into the adding funnel. This solution was added slowly within about 2 h. The reaction mixture was then kept at the same temperature for 5-6 h until the disappearance of the compound (4) was shown by HPLC analysis. A mixture of 20 ml (0.35 mol) of acetic acid and THF (50 mL) was then added drop wise with further vigorous stirring for 10 min. Then 150 mL of water was injected until two clear phases appeared. The organic layer was separated and the aqueous phase was extracted with 4×100 mL of hot toluene (40° C.). The organic layers were combined and THF was removed under vacuum at 45° C. The residual toluene solution was washed with 3×150 mL of water and 2×150 mL of saturated sodium chloride solution, and dried over 8 g MgSO₄ for 1 h. After filtration, the mixture was concentrated under vacuum to furnish 20.5 g of a black, sticky oil. This oil was heated in 40 mL of toluene until complete dissolution, and was subsequently cooled to room temperature (rt). The mixture was kept at this temperature for 10 h, then cooled and kept at 0° C. for 2 h for crystallization. After filtration, the solid was washed by a small amount of toluene and dried at 50° C. for 6 h. The dried solid was dissolved in 15 mL toluene and refluxed with charcoal for 1 h. After filtration, charcoal was washed with toluene. The solutions were combined, cooled to 0° C. and kept at this temperature for 2 h for crystallization. The crystals were isolated by filtration, washed with cold toluene and dried at 50° C. for 8 h under vacuum to afford 2.1 g of the title compound (I) with a purity of >99% (HPLC). All filtrates were combined and concentrated. The residue was purified by column chromatography on silica gel using hexanes/ethyl acetate 10:1 as eluent to afford additional 1.4 g of the title compound (I) with a purity of >98.9% (HPLC). In total 3.5 g (18%) of 5-bromo-7-azaindole (1) were obtained. HPLC retention time of compound 1: 12.307 min.

### Conditions:

Column: Zorbax Eclipse Plus C18, 150 mm*4.6 mm, particle size 3.5 μm

### Column Temperature: 50° C.

### Mobile Phase:

Phase A: 10 mM KH₂PO₃, which was adjusted to pH 2.5 with H₃PO₄

### Phase B: Acetonitrile

### Gradient:

| T (min) | Phase A (%, v/v) | Phase B (%, v/v) |
|---|---|---|
| 0.0 | 95 | 5 |
| 2.0 | 95 | 5 |
| 20.0 | 20 | 80 |
| 25.0 | 20 | 80 |
| 25.1 | 95 | 5 |
| 30.0 | 95 | 5 |

Flow rate: 2.0 mL/min

### Wavelength: UV 230 nm

Injection volume: 5 μl
Preparation of sample solution: A suitable amount of compound 1, about 5.4-6.2 mg, was accurately weighed and dissolved in a 25 mL volumetric flask with solvent by ultrasound.
Solvent: Acetonitrile/H₂O 20:80 (v/v)

### Example 2b

### Synthesis of 5-bromo-7-azaindole (1)

150 mL of LDA (2.0 M in THF, 0.30 mol) was added into a flask and cooled to −30° C. Then 24.2 g (0.10 mol) of the compound of formula (4) was dissolved in 121 mL of dry THF and the solution was slowly added (within about 2 h) to keep the reaction at constant temperature for 5-6 h until the disappearance of the compound (4) was shown by HPLC analysis. Then, 37.2 mL (0.65 mol) of acetic acid in 90 mL of THF was slowly added and the resulting solution was further vigorously stirred for 10 min. Then, 150 mL of water was injected until two clear phases appeared. The organic layer was separated and the aqueous phase was extracted with 4×100 mL of hot toluene (40° C.). The organic phases were combined and THF was removed under vacuum at 45° C. The residual toluene solution was washed with 3×150 mL of water and 2×150 mL of saturated sodium chloride solution, and then dried over 8 g magnesium sulfate for 1 h. After filtration, the mixture was concentrated under vacuum to furnish 22 g of a black, sticky oil. This oil was heated in 40 mL toluene until complete dissolution, and was then cooled to room temperature. The mixture was kept at this temperature for 10 h and then cooled and kept at 0° C. for 2 h for crystallization. After filtration, the solid was washed by a small amount of toluene and dried at 50° C. for 8 h to furnish 2.8 g of the title compound (I) with a HPLC purity of >99%. All filtrates were combined and concentrated. The residue was purified by column chromatography on silica gel using hexanes/ethyl acetate 10:1 as eluent to afford additional 2.2 g of the title compound (I) with a purity of >98.9% (HPLC). In total 5.0 g (26%) of 5-bromo-7-azaindole (1) were obtained.

1. A process for the manufacture of the compound of formula (I) 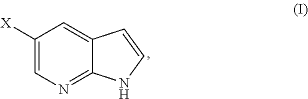
wherein,
(a) the compound of formula (II) 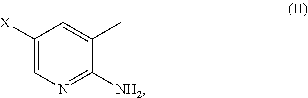
is reacted in the presence of the compound of formula (III) 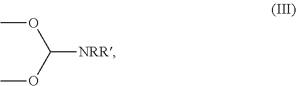
to give the compound of formula (IV) 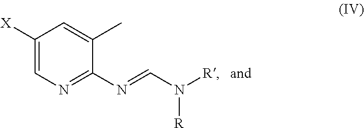
(b) said compound of formula (IV) is further reacted in the presence of a strong base to give the compound of formula (I), and wherein
R and R′ are each independently C1-4 alkyl; and
X is —Cl or —Br.

2. The process according to claim 1, wherein X is —Br.

3. The process according to claim 1, wherein X is —Cl.

4. The process according to claim 2, wherein
R and R′ are both methyl; and
the strong base used in reaction step (b) is lithium diisopropylamide (LDA).

5. The process according to claim 1, wherein
step (a) is carried out using a solvent selected from heptane, toluene, xylene, dimethylsulfoxide (DMSO), dimethylformamide (DMF), chlorobenzene, o-dichlorobenzene, p-dichlorobenzene, m-dichlorobenzene, 2-methyltetrahydrofuran and N-methyl-2-pyrrolidone, and at a temperature within the range of 60 to 120° C.; and
step (b) is carried out using a solvent selected from diethyl ether, methyl t-butyl ether, n-hexane and tetrahydrofuran (THF), a reaction temperature in the range from −85 to 30° C., and a strong base selected from lithium diethylamide, potassium diisopropylamide (KDA), lithium hexamethyldisilazide (LHMDS), sodium hexamethyldisilazide (NaHMDS), n-butyl lithium (n-BuLi), s-Butyl lithium (s-BuLi), t-butyl lithium (t-BuLi), lithium isopropyl cyclohexylamide (LICA), lithium 2,2,6,6-tetramethylpiperidide (TMPLi), ((trimethylsilyl)methyl)lithium (TMSCH₂Li) and lithium di-(trimethylsilyl)methyl amide (TMS₂CHLi).
